(19) United States
(12) Patent Application Publication
(10) Pub. No.: US 2025/0034220 A1
(43) Pub. Date: Jan. 30, 2025

(54) EXPRESSION OF MILK PROTEINS, FAM20C AND RELATED GENES IN PLANTS

(71) Applicant: MOZZA FOODS, Inc., Los Angeles, CA (US)

(72) Inventor: Cory J. Tobin, Los Angeles, CA (US)

(73) Assignee: MOZZA FOODS, Inc., Los Angeles, CA (US)

(21) Appl. No.: 18/786,308

(22) Filed: Jul. 26, 2024

Related U.S. Application Data
(60) Provisional application No. 63/516,115, filed on Jul. 27, 2023.
(60) Provisional application No. 63/517,532, filed on Aug. 3, 2023.

(51) Int. Cl. C07K 14/47; A23L 33/19

(52) U.S. Cl. CPC C07K 14/4732; A23L 33/19; C07K 2319/00

(57) ABSTRACT

Compositions for expression of milk proteins in plants and co-expression of milk proteins and kinases in plants are disclosed herein. With respect to the disclosed composition herein, nucleic acids, which encode for the milk proteins and kinases, can be isolated. The milk proteins, which are expressed or co-expressed with kinases, in the plants, include: alpha S1 casein, alpha S2 casein, beta casein, and kappa casein. The kinases, which are co-expressed with the milk proteins, include: FAM20C and FAM20A.

### CROSS-REFERENCE

This application claims the benefit of U.S. Provisional Application Ser. No. 63/516,115, filed on Jul. 27, 2023, and U.S. Provisional Application Ser. No. 63/517,532, filed on Aug. 3, 2023, which are each incorporated herein by reference in its entirety.

### INCORPORATION BY REFERENCE

The present application is being filed along with a Sequence Listing in electronic format. The Sequence Listing is provided as a file entitled 716100USNP_SEQ_LIST.xml, created on Jul. 26, 2024, which is 1,480 kilobytes in size. The information in the electronic format of the Sequence Listing is incorporated by reference in its entirety.

### BACKGROUND

Casein micelles account for more than 80% of the protein in bovine milk and are a key component of all dairy cheeses. Casein micelles include individual casein proteins are produced in the mammary glands of bovines and other ruminants. The industrial scale production of the milk that is processed to yield these casein micelles, primarily in the form of curds for cheese production, typically occurs on large-scale dairy farms and is often inefficient, damaging to the environment, and harmful to the animals. Dairy cows contribute substantially to greenhouse gasses, consume significantly more water than the milk they produce, and commonly suffer from dehorning, disbudding, mastitis, routine forced insemination, and bobby calf slaughter.

Accordingly, there is a need for an in vivo plant-based casein expression system which allows for purification of biologically active casein proteins that is cost effective at industrial scale. A major impediment in making casein proteins and other milk proteins is the difficulty of expressing those proteins in plants. Solutions to these problems have been long sought but prior developments have not taught or suggested any solutions and, thus, solutions to these problems have long eluded those skilled in the art.

### SUMMARY

Some aspects of the disclosure provide nucleotide sequences for expressing milk proteins, for example, casein proteins, in plants and food products that comprise the nucleotide sequences.

Some aspects of the disclosure, with respect to expressing milk proteins, such as caseins, in plants, a nucleic acid molecule comprising a nucleotide sequence at least 85%, at least 90%, at least 95%, at least 96%, at least 97%, at least 98%, at least 99%, or 100% identical to any one of SEQ ID NO:1-SEQ ID NO:200. In some cases, the nucleotide sequence is between 72% and 80% identical to SEQ ID NO: 201. In some cases, the nucleotide sequence codes for alpha S1 casein. In some cases, the alpha S1 casein is a bovine alpha S1 casein.

Some aspects of the disclosure, with respect to expressing milk proteins, such as caseins, in plants, a nucleic acid molecule comprising a nucleotide sequence at least 85%, at least 90%, at least 95%, at least 96%, at least 97%, at least 98%, at least 99%, or 100% identical to any one of SEQ ID NO:202-SEQ ID NO:400. In some cases, the nucleotide sequence is between 72% and 80% identical to SEQ ID NO: 401. In some cases, the nucleotide sequence codes for alpha S2 casein. In some cases, the alpha S2 casein is a bovine alpha S2 casein.

Some aspects of the disclosure, with respect to expressing milk proteins, such as caseins, in plants, a nucleic acid molecule comprising a nucleotide sequence at least 85%, at least 90%, at least 95%, at least 96%, at least 97%, at least 98%, at least 99%, or 100% identical to any one of SEQ ID NO:402-SEQ ID NO:603. In some cases, the nucleotide sequence is between 72% and 80% identical to SEQ ID NO: 604. In some cases, the nucleotide sequence codes for beta casein. In some cases, the beta casein is a bovine beta casein.

Some aspects of the disclosure, with respect to expressing milk proteins, such as caseins, in plants, a nucleic acid molecule comprising a nucleotide sequence at least 85%, at least 90%, at least 95%, at least 96%, at least 97%, at least 98%, at least 99%, or 100% identical to any one of SEQ ID NO:605-SEQ ID NO:805. In some cases, the nucleotide sequence is between 72% and 80% identical to SEQ ID NO: 806. In some cases, the nucleotide sequence codes for kappa casein. In some cases, the kappa casein is a bovine kappa casein.

In some cases, with respect to expressing milk proteins, such as caseins, in plants, the nucleic acid molecule comprises a nucleotide sequence that is at least 70% but no more than 80% identical to any one of SEQ ID NO:1-SEQ ID NO:806. In some cases, the nucleic acid molecule comprises a nucleotide sequence that is at least 72% but no more than 78% identical to any one of SEQ ID NO:1-SEQ ID NO:806. In some cases, the nucleic acid molecule comprises a nucleotide sequence that is at least 74% but no more than 76% identical to any one of SEQ ID NO:1-SEQ ID NO: 806. In some cases, the nucleic acid molecule codes for alpha S1 casein, alpha S2 casein, beta casein, or kappa casein. In some cases, the nucleic acid is isolated.

In some cases, with respect to expressing milk proteins, such as caseins, in plants, the disclosed the nucleic acid does not comprise at least two, at least three, at least four, at least five, at least six, at least seven, at least eight, at least nine, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, or at least 46 of the sequences selected from the group consisting of GGTACC, ACCGGT, GGGCCC, GTGCAC, GGCGCGCC, GGTACC, CCTAGG, GGATCC, AGATCT, CACGTC, ATCGAT, TTCGAA, ATCGAT, CGGCCG, GAGCTC, GAATTC, GATATC, AAGCTT, GTTAAC, GGTACC, ACGCGT, CCATGG, CATATG, GCTAGC, GCGGCCGC, ATGCAT, TTAATTAA, CTCGAG, GGGCCC, CTGCAG, CGATCG, CAGCTG, GAGCTC, CCGCGG, GTCGAC, CCCGGG, TACGTA, ACTAGT, GCATGC, CTCGAG, CCCGGG, TCTAGA, CTCGAG, CCCGGG, GGTCTC and GAAGAC. In some cases, the disclosed nucleic acid molecule does not comprise GGTCTC. In some cases, the disclosed nucleic acid molecule does not comprise GAAGAC.

Some aspects of the disclosure, with respect to expressing milk proteins, such as caseins, in plants, provide a nucleic acid molecule comprising a nucleotide sequence at least 85%, at least 90%, at least 95%, at least 96%, at least 97%, at least 98%, at least 99%, or 100% identical to any one of SEQ ID NO:1-SEQ ID NO:200, wherein the nucleotide sequence is between 72% and 80% identical to SEQ ID NO: 201.

Some aspects of the disclosure, with respect to expressing milk proteins, such as caseins, in plants, provide a nucleic acid molecule comprising a nucleotide sequence at least 85%, at least 90%, at least 95%, at least 96%, at least 97%, at least 98%, at least 99%, or 100% identical to any one of SEQ ID NO:202-SEQ ID NO:400, wherein the nucleotide sequence is between 72% and 80% identical to SEQ ID NO: 401.

Some aspects of the disclosure, with respect to expressing milk proteins, such as caseins, in plants, provide a nucleic acid molecule comprising a nucleotide sequence at least 85%, at least 90%, at least 95%, at least 96%, at least 97%, at least 98%, at least 99%, or 100% identical to any one of SEQ ID NO:402-SEQ ID NO:603, wherein the nucleotide sequence is between 72% and 80% identical to SEQ ID NO: 604.

Some aspects of the disclosure, with respect to expressing milk proteins, such as caseins, in plants, provide a nucleic acid molecule comprising a nucleotide sequence at least 85%, at least 90%, at least 95%, at least 96%, at least 97%, at least 98%, at least 99%, or 100% identical to any one of SEQ ID NO:605-SEQ ID NO:805, wherein the nucleotide sequence is between 72% and 80% identical to SEQ ID NO: 806.

Some aspects of the disclosure, with respect to expressing milk proteins, such as caseins, in plants, provide a food composition comprising any one of the nucleic acid molecules disclosed herein. In some cases, the food composition is a dairy product. In some cases, the food composition comprises a plant molecule. In some cases, the plant molecule is a plant protein, sugar or deoxyribonucleic acid. In some cases, the food composition is cheese.

Some aspects of the disclosure, with respect to expressing milk proteins, such as caseins, in plants, provide a fusion protein, comprising a casein protein and a peptide sequence, wherein the peptide sequence comprises an amino acid sequence that is at least 85%, at least 90%, at least 95%, at least 96%, at least 97%, at least 98%, at least 99%, or 100% identical to SEQ ID NO: 808. In some cases, the casein protein is alpha S1 casein, alpha S2 casein, beta casein, or kappa casein.

Some aspects of the disclosure, with respect to expressing milk proteins, such as caseins, in plants, provide a nucleic acid molecule comprising a first nucleotide sequence that is at least 85%, at least 90%, at least 95%, at least 96%, at least 97%, at least 98%, at least 99%, or 100% identical to any one of SEQ ID NO:1-SEQ ID NO: 806, and a second nucleotide sequence that is at least 85%, at least 90%, at least 95%, at least 96%, at least 97%, at least 98%, at least 99%, or 100% identical SEQ ID NO: 807.

In some aspects, the nucleotide sequences, with respect to expressing milk proteins, such as caseins, in plants, provide codon-optimized for a plant, wherein the plant can be any one of the following, for example, angiosperms and gymnosperms such as Arabidopsis, potato, tomato, tobacco, alfalfa, lemice, carrot, strawberry, sugarbeet, cassava, sweet potato, soybean, lima bean, pea, chick pea, maize (com), turf grass, wheat, rice, barley, sorghum, oat, oak, eucalyptus, walnut, palm, and duckweed a well as fern and moss. In some aspects, the nucleotide sequences provided herein are codon-optimized for a plant, wherein the plant is a monocot, a dicot, or a vascular plant reproduced from spores such as fern or a nonvascular plant such as moss, liverwort, hornwort, and algae. In some aspects, the nucleotide sequences provided herein are codon-optimized for a dicot plant, include for example Arabidopsis, tobacco, tomato, potato, sweet potato, cassava, alfalfa, lima bean, pea, chick pea, soybean, carrot, strawberry, lettuce, oak, maple, walnut, rose, mint, squash, daisy, quinoa, buckwheat, mung bean, cow pea, lentil, lupin, peanut, fava bean, French beans, mustard, or cactus. In some aspects, the nucleotide sequences provided herein are codon-optimized for a monocot plant, including for example, turf grass, maize (corn), rice, oat, wheat, barley, sorghum, orchid, iris, lily, onion, palm, and duckweed. In some cases, the nucleotide sequences provided herein are codon-optimized for a soybean (i.e., glycine max).

In some cases, the nucleotide sequences, with respect to expressing milk proteins, such as caseins, in plants, do not comprise a nucleotide sequence that is susceptible to enzymatic digestion by one or more restriction enzymes. For example, the nucleotide sequences provided herein do not comprise a nucleotide sequence that is susceptible to enzymatic digestion by one or more the following restriction digestion enzymes: Acc65I (GGTACC), AgeI (ACCGGT), ApaI (GGGCCC), ApaLI (GTGCAC), AscI (GGCGCGCC), Asp718I (GGTACC), AvrII (CCTAGG), BamHI (GGATCC), BglII (AGATCT), BmgBI (CACGTC), BspDI (ATCGAT), BstBI (TTCGAA), ClaI (ATCGAT), EagI (CGGCCG), Ecl136II (GAGCTC), EcoRI (GAATTC), EcoRV (GATATC), HindIII (AAGCTT), HpaI (GTTAAC), KpnI (GGTACC), MluI (ACGCGT), NcoI (CCATGG), NdeI (CATATG), NheI (GCTAGC), NotI (GCGGCCGC), NsiI (ATGCAT), PacI (TTAATTAA), PaeR7I (CTCGAG), PspOMI (GGGCCC), PstI (CTGCAG), PvuI (CGATCG), PvuII (CAGCTG), SacI (GAGCTC), SacII (CCGCGG), SalI (GTCGAC), SmaI (CCCGGG), SnaBI (TACGTA), SpeI (ACTAGT), SphI (GCATGC), TliI (CTCGAG), TspMI (CCCGGG), XbaI (TCTAGA), XhoI (CTCGAG), XmaI (CCCGGG). In some cases, the nucleotide sequences provided herein do not comprise one or more of the nucleotide sequences in the list above. In some cases, the nucleotide sequences provided herein do not comprise any of the nucleotide sequences in the list above. In some cases, the nucleotide sequences provided herein do not comprise a nucleotide sequence that is susceptible to enzymatic digestion by Eco31I (i.e., BsaI, Bso31I, BspTNI) or BpiI (i.e., BbsI, BpuAI, BstV2I). In some cases, the nucleotide sequences provided herein do not comprise nucleotide sequences GGTCTC or GAAGAC.

Some aspects of the disclosure provide nucleotide sequences for co-expressing milk protein with a kinase (for example, FAM20C) to improve the expression and function of milk protein in plants and food products that comprise the nucleotide sequences.

Some aspects of the disclosure, with respect to expressing milk proteins and kinases in plants, a nucleic acid molecule comprising a nucleotide sequence at least 85%, at least 90%, at least 95%, at least 96%, at least 97%, at least 98%, at least 99%, or 100% identical to any one of SEQ ID NO: 814-SEQ ID NO: 920. In some cases, the nucleotide sequence is between 74% and 78% identical to SEQ ID NO: 921. In some cases, the nucleotide sequence is between 74% and 77% identical to SEQ ID NO: 921. In some cases, the nucleotide sequence is between 74% and 76% identical to SEQ ID NO: 921. In some cases, the nucleotide sequence is between 74% and 75% identical to SEQ ID NO: 921. In some cases, the nucleotide sequence is between 75% and 78% identical to SEQ ID NO: 921. In some cases, the nucleotide sequence is between 75% and 77% identical to SEQ ID NO: 921. In some cases, the nucleotide sequence is between 75% and 76% identical to SEQ ID NO: 921. In some cases, the nucleotide sequence is between 76% and 78% identical to SEQ ID NO: 921. In some cases, the nucleotide sequence is between 76% and 77% identical to SEQ ID NO: 921. In some cases, the nucleotide sequence is between 76% and 78% identical to SEQ ID NO: 921. In some cases, the nucleotide sequence is between 76% and 77% identical to SEQ ID NO: 921. In some cases, the nucleotide sequence is between 77% and 78% identical to SEQ ID NO: 921. In some cases, the nucleotide sequence codes for FAM20C. In some cases, FAM20C is a bovine protein.

Some aspects of the disclosure, with respect to co-expressing milk proteins with kinases, a nucleic acid molecule comprising a nucleotide sequence at least 85%, at least 90%, at least 95%, at least 96%, at least 97%, at least 98%, at least 99%, or 100% identical to any one of SEQ ID NO: 922-SEQ ID NO: 926. In some cases, the nucleotide sequence codes for FAM20A. In some cases, FAM20A is a bovine protein.

In some cases of the disclosure, with respect to co-expressing milk proteins with kinases, the disclosed the nucleic acid does not comprise at least two, at least three, at least four, at least five, at least six, at least seven, at least eight, at least nine, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, or at least 46 of the sequences selected from the group consisting of GGTACC, ACCGGT, GGGCCC, GTGCAC, GGCGCGCC, GGTACC, CCTAGG, GGATCC, AGATCT, CACGTC, ATCGAT, TTCGAA, ATCGAT, CGGCCG, GAGCTC, GAATTC, GATATC, AAGCTT, GTTAAC, GGTACC, ACGCGT, CCATGG, CATATG, GCTAGC, GCGGCCGC, ATGCAT, TTAATTAA, CTCGAG, GGGCCC, CTGCAG, CGATCG, CAGCTG, GAGCTC, CCGCGG, GTCGAC, CCCGGG, TACGTA, ACTAGT, GCATGC, CTCGAG, CCCGGG, TCTAGA, CTCGAG, CCCGGG, GGTCTC and GAAGAC. In some cases, the disclosed nucleic acid molecule does not comprise GGTCTC. In some cases, the disclosed nucleic acid molecule does not comprise GAAGAC.

Some aspects of the disclosure, with respect to co-expressing milk proteins with kinases, provide a plant comprising any one of the nucleic acid molecules in any one of SEQ ID NO: 814-SEQ ID NO: 920. In some aspects, the nucleotide sequences provided herein are codon-optimized for a plant, wherein the plant can be any one of the following, for example, angiosperms and gymnosperms such as Arabidopsis, potato, tomato, tobacco, alfalfa, lemice, carrot, strawberry, sugarbeet, cassava, sweet potato, soybean, lima bean, pea, chick pea, maize (com), turf grass, wheat, rice, barley, sorghum, oat, oak, eucalyptus, walnut, palm and duckweed a well as fern and moss. In some aspects, the nucleotide sequences provided herein are codon-optimized for a plant, wherein the plant is a monocot, a dicot, or a vascular plant reproduced from spores such as fern or a nonvascular plant such as moss, liverwort, hornwort and algae. In some aspects, the nucleotide sequences provided herein are codon-optimized for a dicot plant, include for example Arabidopsis, tobacco, tomato, potato, sweet potato, cassava, alfalfa, lima bean, pea, chick pea, soybean, carrot, strawberry, lettuce, oak, maple, walnut, rose, mint, squash, daisy, quinoa, buckwheat, mung bean, cow pea, lentil, lupin, peanut, fava bean, French beans, mustard, or cactus. In some aspects, the nucleotide sequences provided herein are codon-optimized for a monocot plant, including for example, turf grass, maize (corn), rice, oat, wheat, barley, sorghum, orchid, iris, lily, onion, palm, and duckweed. In some cases, the nucleotide sequences provided herein are codon-optimized for a soybean (i.e., glycine max).

In some cases of the disclosure, with respect to co-expressing milk proteins with kinases, the nucleotide sequences provided herein do not comprise a nucleotide sequence that is susceptible to enzymatic digestion by one or more restriction enzymes. For example, the nucleotide sequences provided herein do not comprise a nucleotide sequence that is susceptible to enzymatic digestion by one or more the following restriction digestion enzymes: Acc65I (GGTACC), AgeI (ACCGGT), ApaI (GGGCCC), ApaLI (GTGCAC), AscI (GGCGCGCC), Asp718I (GGTACC), AvrII (CCTAGG), BamHI (GGATCC), BglII (AGATCT), BmgBI (CACGTC), BspDI (ATCGAT), BstBI (TTCGAA), ClaI (ATCGAT), EagI (CGGCCG), Ecl136II (GAGCTC), EcoRI (GAATTC), EcoRV (GATATC), HindIII (AAGCTT), HpaI (GTTAAC), KpnI (GGTACC), MluI (ACGCGT), NcoI (CCATGG), NdeI (CATATG), NheI (GCTAGC), NotI (GCGGCCGC), NsiI (ATGCAT), PacI (TTAATTAA), PacR7I (CTCGAG), PspOMI (GGGCCC), PstI (CTGCAG), PvuI (CGATCG), PvuII (CAGCTG), SacI (GAGCTC), SacII (CCGCGG), SalI (GTCGAC), SmaI (CCCGGG), SnaBI (TACGTA), SpeI (ACTAGT), SphI (GCATGC), TliI (CTCGAG), TspMI (CCCGGG), XbaI (TCTAGA), XhoI (CTCGAG), XmaI (CCCGGG). In some cases, the nucleotide sequences provided herein do not comprise one or more of the nucleotide sequences in the list above. In some cases, the nucleotide sequences provided herein do not comprise any of the nucleotide sequences in the list above. In some cases, the nucleotide sequences provided herein do not comprise a nucleotide sequence that is susceptible to enzymatic digestion by Eco31I (i.e., BsaI, Bso31I, BspTNI) or BpiI (i.e., BbsI, BpuAI, BstV21). In some cases, the nucleotide sequences provided herein do not comprise nucleotide sequences GGTCTC or GAAGAC.

In some embodiments of the disclosure, with respect to co-expressing milk proteins with kinases, the provided DNA sequences contribute to new protein folding patterns compared to wild type, due to the impact of codon changes on the rate of protein synthesis. In some embodiments, changes in codon usage enhance the stability and localization of mRNA, optimizing the quantity and positioning of protein synthesis. In some embodiments, the variability of tRNA availability across organisms aligns with the introduced codons, prompting unique translation dynamics. In some embodiments, the modified codon usage enriches traditional patterns of gene expression by affecting the regulatory sequences of DNA and RNA. In some embodiments, changes in codon usage provide an additional dimension to post-translational modifications vital to protein function by introducing alterations in translation speed and timing.

Some aspects of the disclosure, with respect to co-expressing milk proteins with kinases, provide a composition comprising any one of the nucleic acid molecules disclosed herein. In some cases, the composition is a food composition. In some cases, the food composition is a dairy product. In some cases, the food composition comprises a plant molecule. In some cases, the plant molecule is a plant protein, sugar or deoxyribonucleic acid. In some cases, the food composition is cheese.

Some aspects of the disclosure. with respect to co-expressing milk proteins with kinases, provide a food composition comprising any one of the nucleic acid molecules in SEQ ID NO: 922-SEQ ID NO: 926. Some aspects of the disclosure provide a food composition comprising: a casein protein or a DNA coding for a casein protein; and a nucleic acid molecule in any one of SEQ ID NO:814-SEQ ID NO:926. Some aspects of the disclosure provide a food composition comprising: a first nucleic acid molecule in any one of SEQ ID NO: 814-SEQ ID NO: 920; and a second nucleic acid molecule in any one of SEQ ID NO: 922-SEQ ID NO: 926. In some cases, the food composition further comprises a casein protein or a DNA coding for a casein protein. In some cases, the food composition is a dairy product. In some cases, the food composition comprises a plant molecule. In some cases, the plant molecule is a plant protein, sugar, or deoxyribonucleic acid.

In some cases of the disclosure, with respect to co-expressing milk proteins with kinases, the composition does not comprise a detectable amount of α-lactalbumin. In some cases, the composition does not comprise a detectable amount of β-lactoglobulin. In some cases, the composition does not comprise a detectable amount of α-S2-casein. In some cases, the composition does not comprise a detectable amount of lactoferrin. In some cases, the composition does not comprise a detectable amount of transferrin. In some cases, the composition does not comprise a detectable amount of serum albumin. In some cases, the composition does not comprise a detectable amount of lysozyme. In some cases, the composition does not comprise a detectable amount of lactoperoxidase. In some cases, the composition does not comprise a detectable amount of immunoglobulin-A. In some cases, the composition does not comprise a detectable amount of lipase.

In some cases of the disclosure, with respect to co-expressing milk proteins with kinases, the composition is free of α-lactalbumin. In some cases, the composition is free of β-lactoglobulin. In some cases, the composition is free of α-S2-casein. In some cases, the composition is free of α-S1-casein. In some cases, the composition is free of β-casein. In some cases, the composition is free of lactoferrin. In some cases, the composition is free of transferrin. In some cases, the composition is free of serum albumin. In some cases, the composition is free of lysozyme. In some cases, the composition is free of lactoperoxidase. In some cases, the composition is free of immunoglobulin-A. In some cases, the composition is free of lipase.

In some cases, the composition, with respect to co-expressing milk proteins with kinases, is essentially free of α-lactalbumin. In some cases, the composition is essentially free of β-lactoglobulin. In some cases, the composition is essentially free of α-S2-casein. In some cases, the composition is essentially free of β-casein. In some cases, the composition is essentially free of α-S1-casein. In some cases, the composition is essentially free of lactoferrin. In some cases, the composition is essentially free of transferrin. In some cases, the composition is essentially free of serum albumin. In some cases, the composition is essentially free of lysozyme. In some cases, the composition is essentially free of lactoperoxidase. In some cases, the composition is essentially free of immunoglobulin-A. In some cases, the composition is essentially free of lipase.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DETAILED DESCRIPTION

The following embodiments are described in sufficient detail to enable those skilled in the art to make and use the invention. It is to be understood that other embodiments would be evident based on the present disclosure, and that system, process, or mechanical changes can be made without departing from the scope of an embodiment of the present disclosure.

In the following description, numerous specific details are given to provide a thorough understanding of the invention. However, it will be apparent that the invention can be practiced without these specific details. In order to avoid obscuring an embodiment of the present disclosure, some well-known techniques, system configurations, and process steps are not disclosed in detail. Throughout this disclosure, various publications, patents, and published patent specifications are referenced by an identifying citation. The disclosures of these publications, patents and published patent specifications are hereby incorporated by reference into the present disclosure.

In the compositions herein, nucleotide sequences for: (i) expressing milk proteins comprising: alpha S1 casein; alpha S2 casein; beta casein; and kappa casein, in plants and food products that comprise the nucleotide sequences for expressing casein are disclosed; and (ii) nucleotide sequences for co-expressing one or more of the milk proteins comprising: alpha S1 casein; alpha S2 casein; beta casein; and kappa casein, with a kinase comprising FAM20C and FAMC20A, to improve the expression and function of milk protein in plants and food products that comprise the nucleotide sequences.

The compositions herein comprise SEQ ID NO: 1-926, which can be nucleic acids that are isolated, codon optimized, and integrated into a plant cell, OR a fusion protein, wherein: SEQ ID NO: 1-SEQ ID NO: 200 and SEQ ID NO: 809 are each a nucleotide sequence encoding for alpha S1 casein; SEQ ID NO: 201 is a nucleotide sequence encoding for wild type alpha S1 casein; SEQ ID NO: 202-SEQ ID NO: 400 and SEQ ID NO: 810 are each a nucleotide sequence encoding for alpha S2 casein; SEQ ID NO: 401 is a nucleotide sequence encoding for wild type alpha S2 casein; SEQ ID NO: 402-SEQ ID NO: 603 and SEQ ID NO: 811 are each a nucleotide sequence encoding for beta casein; SEQ ID NO: 604 is nucleotide sequence encoding for wild type beta casein; SEQ ID NO: 605-SEQ ID NO: 805 and SEQ ID NO: 812 are each a nucleotide sequence encoding for kappa casein; SEQ ID NO: 806 is nucleotide sequence encoding for wild type kapa casein; SEQ ID NO: 807 is a nucleotide sequence encoding for GY1 10AA VSD; SEQ ID NO: 808 is a peptide sequence, GY1 10AA VSD, combined with said casein protein or said casein proteins to yield a fusion protein; SEQ ID NO: 813 is a nucleotide sequence encoding for FIMXQ3; and SEQ ID NO: 814-SEQ ID NO: 920 are each a nucleotide sequence encoding for FAM20C; SEQ ID NO: 921 is nucleotide sequence encoding for wild type FAM20C; and SEQ ID NO: 922-Seq ID NO: 926 are each a nucleotide sequence encoding for FAM20A.

In the compositions herein, an isolated nucleotide sequence can be: (i) 72-80% identical to a wild type variant, such as SEQ ID NO: 201, SEQ ID NO:401, SEQ ID NO: 604, and SEQ ID NO: 806; (ii) between 74% and 78%, 74% and 77%, 74%-76%, 74%-75%, 75%-78%, 75%-77%, 75%-76%, 76%-78%, 76%-77%, or 77%-78% identical to SEQ ID NO: 921.

In the compositions herein, an isolated nucleotide sequence can be at least 85%, at least 90%, at least 95%, at least 96%, at least 97%, at least 98%, at least 99%, or 100% identical to SEQ ID NO: 1-SEQ ID NO: 200 and SEQ ID NO: 809; SEQ ID NO: 202-SEQ ID NO: 400 and SEQ ID NO: 810; SEQ ID NO: 402-SEQ ID NO: 603 and SEQ ID NO: 811; SEQ ID NO: 605-SEQ ID NO: 805 and SEQ ID NO: 812; SEQ ID NO: 813; SEQ ID NO: 814-SEQ ID NO: 920; and SEQ ID NO: 922-926.

In the compositions herein, SEQ ID NO: 1-SEQ ID NO: 807 and SEQ ID NO: 809-SEQ ID NO: 926 can be nucleic acids that do not comprise at least two, at least three, at least four, at least five, at least six, at least seven, at least eight, at least nine, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, or at least 46 of the sequences selected from the group consisting of GGTACC, ACCGGT, GGGCCC, GTGCAC, GGCGCGCC, GGTACC, CCTAGG, GGATCC, AGATCT, CACGTC, ATCGAT, TTCGAA, ATCGAT, CGGCCG, GAGCTC, GAATTC, GATATC, AAGCTT, GTTAAC, GGTACC, ACGCGT, CCATGG, CATATG, GCTAGC, GCGGCCGC, ATGCAT, TTAATTAA, CTCGAG, GGGCCC, CTGCAG, CGATCG, CAGCTG, GAGCTC, CCGCGG, GTCGAC, CCCGGG, TACGTA, ACTAGT, GCATGC, CTCGAG, CCCGGG, TCTAGA, CTCGAG, CCCGGG, GGTCTC and GAAGAC.

In the compositions herein, SEQ ID NO: 1-807 and SEQ ID NO: 809-926 can be nucleic acids that do not comprise GGTCTC or GAAGAC.

In the compositions herein, a fusion protein can comprise a casein protein and a peptide sequence, wherein the peptide sequence comprises an amino acid sequence that is at least 85%, at least 90%, at least 95%, at least 96%, at least 97%, at least 98%, at least 99%, or 100% identical to SEQ ID NO: 808, wherein the casein protein is alpha-S1 casein, alpha-S2 casein, beta casein, or kappa casein.

In the compositions herein, a nucleic acid molecule can comprise: a first nucleotide sequence that is at least 85%, at least 90%, at least 95%, at least 96%, at least 97%, at least 98%, at least 99%, or 100% identical to any one of SEQ ID NO:1-SEQ ID NO: 806, and a second nucleotide sequence that is at least 85%, at least 90%, at least 95%, at least 96%, at least 97%, at least 98%, at least 99%, or 100% identical SEQ ID NO: 807.

In the compositions herein, SEQ ID NO: 1-SEQ ID NO: 926 can be used, and therefore comprised within a food composition, such as a dairy product, which contain one or more plant molecules selected from a plant protein, sugar, or deoxyribonucleic acid.

In the compositions herein, SEQ ID NO: 1-SEQ ID NO: 926 can be used for the expression of caseins (alpha-S1 casein, alpha-S2 casein, beta casein, and kappa casein) and kinases (FAM20C and FAM20A) in plants, wherein said caseins and kinases can be bovine, buffalo, goat, sheep and camels, yaks, horses, reindeers and donkey proteins.

With respect to co-expressing milk proteins with kinases, the compositions herein can be free of α-lactalbumin or β-lactoglobulin; free of α-S2-casein, free of α-S1-casein, β-casein; free of lactoferrin; free of transferrin; free of serum album; free of lysozyme; free of lactoperoxidase; free of immunoglobulin-A; and/or free of lipase.

With respect to co-expressing milk proteins with kinases, the compositions herein can be essentially free of or not comprise a detectable amount of α-lanctalbumin or β-lactoglobulin; essentially free of or not comprise a detectable amount of α-S2-casein, essentially free of or not comprise a detectable amount of α-S1-casein, essentially free of or not comprise a detectable amount of β-casein; essentially free of or not comprise a detectable amount of lactoferrin; essentially free of or not comprise a detectable amount of transferrin; essentially free of or not comprise a detectable amount of serum album; essentially free of or not comprise a detectable amount of lysozyme; essentially free of or not comprise a detectable amount of lactoperoxidase; essentially free of or not comprise a detectable amount of immunoglobulin-A; and/or essentially free of or not comprise a detectable amount of lipase. Compositions essentially free of these said proteins have trace amounts that are higher than the amounts of these proteins when the composition is free of these said proteins.

With respect to co-expressing milk proteins with kinases, the nucleotide sequences, such as SEQ ID NO: 814-SEQ ID NO: 926, as provided herein, do not comprise a nucleotide sequence that is susceptible to enzymatic digestion by one or more restriction enzymes. For example, the nucleotide sequences provided herein do not comprise a nucleotide sequence that is susceptible to enzymatic digestion by one or more the following restriction digestion enzymes: Acc65I (GGTACC), AgeI (ACCGGT), ApaI (GGGCCC), ApaLI (GTGCAC), AscI (GGCGCGCC), Asp718I (GGTACC), AvrII (CCTAGG), BamHI (GGATCC), BglII (AGATCT), BmgBI (CACGTC), BspDI (ATCGAT), BstBI (TTCGAA), ClaI (ATCGAT), EagI (CGGCCG), Ecl136II (GAGCTC), EcoRI (GAATTC), EcoRV (GATATC), HindIII (AAGCTT), HpaI (GTTAAC), KpnI (GGTACC), MluI (ACGCGT), NcoI (CCATGG), NdeI (CATATG), NheI (GCTAGC), NotI (GCGGCCGC), NsiI (ATGCAT), PacI (TTAATTAA), PacR7I (CTCGAG), PspOMI (GGGCCC), PstI (CTGCAG), PvuI (CGATCG), PvuII (CAGCTG), SacI (GAGCTC), SacII (CCGCGG), SalI (GTCGAC), SmaI (CCCGGG), SnaBI (TACGTA), SpeI (ACTAGT), SphI (GCATGC), TliI (CTCGAG), TspMI (CCCGGG), XbaI (TCTAGA), XhoI (CTCGAG), XmaI (CCCGGG). In some cases, the nucleotide sequences provided herein do not comprise one or more of the nucleotide sequences in the list above. In some cases, the nucleotide sequences provided herein do not comprise any of the nucleotide sequences in the list above. In some cases, the nucleotide sequences provided herein do not comprise a nucleotide sequence that is susceptible to enzymatic digestion by Eco31I (i.e., BsaI, Bso31I, BspTNI) or BpiI (i.e., BbsI, BpuAI, BstV2I). In some cases, the nucleotide sequences provided herein do not comprise nucleotide sequences GGTCTC or GAAGAC.

The compositions herein, which include the nucleotide sequences SEQ ID NO: 1-SEQ ID NO: 807, and SEQ ID NO: 809-SEQ ID NO: 926, can be codon-optimized for a plant, wherein the plant can be any one of the following, for example, angiosperms and gymnosperms such as Arabidopsis, potato, tomato, tobacco, alfalfa, lemice, carrot, strawberry, sugar beet, cassava, sweet potato, soybean, lima bean, pea, chick pea, maize (com), turf grass, wheat, rice, barley, sorghum, oat, oak, eucalyptus, walnut, palm and duckweed a well as fern and moss. In some aspects, the nucleotide sequences provided herein are codon-optimized for a plant, wherein the plant is a monocot, a dicot, or a vascular plant reproduced from spores such as fern or a nonvascular plant such as moss, liverwort, hornwort and algae. In some aspects, the nucleotide sequences provided herein are codon-optimized for a dicot plant, include for example Arabidopsis, tobacco, tomato, potato, sweet potato, cassava, alfalfa, lima bean, pea, chick pea, soybean, carrot, strawberry, lettuce, oak, maple, walnut, rose, mint, squash, daisy, quinoa, buckwheat, mung bean, cow pea, lentil, lupin, peanut, fava bean, French beans, mustard, or cactus. In some aspects, the nucleotide sequences provided herein are codon-optimized for a monocot plant, including for example, turf grass, maize (corn), rice, oat, wheat, barley, sorghum, orchid, iris, lily, onion, palm, and duckweed. In some cases, the nucleotide sequences provided herein are codon-optimized for a soybean (i.e., glycine max).

In the compositions herein, SEQ ID NO: 1-SEQ ID NO: 807 and SEQ ID NO: 809-SEQ ID NO: 926 can contribute to new protein folding patterns compared to wild type, due to the impact of codon changes on the rate of protein synthesis. In some embodiments, changes in codon usage enhance the stability and localization of mRNA, optimizing the quantity and positioning of protein synthesis. In some embodiments, the variability of tRNA availability across organisms aligns with the introduced codons, prompting unique translation dynamics. In some embodiments, the modified codon usage enriches traditional patterns of gene expression by affecting the regulatory sequences of DNA and RNA. In some embodiments, changes in codon usage provide an additional dimension to post-translational modifications vital to protein function by introducing alterations in translation speed and timing.

In the compositions herein, a food composition (such as a dairy product) can comprise: a casein protein or a DNA coding for a casein protein comprising SEQ ID NO: 1-SEQ ID NO: 807 or SEQ ID NO: 809-SEQ ID NO: 813; and a nucleic acid molecule comprising SEQ ID NO: 814-SEQ ID NO: 926.

In the compositions herein, a food composition (such as a dairy product) can comprise: a first nucleic acid molecule comprising SEQ ID NO: 814-SEQ ID NO: 926; and a second nucleic acid molecule comprising SEQ ID NO: 814-SEQ ID NO: 926.

### Definitions

These and other valuable aspects of the embodiments of the present disclosure consequently further the state of the technology to at least the next level. While the disclosure has been described in conjunction with a specific best mode, it is to be understood that many alternatives, modifications, and variations will be apparent to those skilled in the art in light of the descriptions herein. Accordingly, it is intended to embrace all such alternatives, modifications, and variations that fall within the scope of the included claims. All matters set forth herein or shown in the accompanying drawings are to be interpreted in an illustrative and non-limiting sense.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular and not limited to sequential steps. Accordingly, terms such as “first” and “second” do not necessarily imply priority, order of importance, or order of acts.

As used herein, the term “about” or the symbol “˜” when referring to a number or a numerical range means that the number or numerical range referred to is an approximation within experimental variability (or within statistical experimental error), and the number or numerical range may vary from, for example, from 1% to 10% of the stated number or numerical range. Unless otherwise indicated by context, the term “about” refers to +10% of a stated number or value.

As used herein, the term “approximately” means within an acceptable error range for the particular value as determined by one of ordinary skill in the art, which will depend in part on how the value is measured or determined, e.g., the limitations of the measurement system. For example, “approximately” can mean within 1 or more than 1 standard deviation, per the practice in the given value. Where particular values are described in the application and claims, unless otherwise stated the term “approximately” should be assumed to mean an acceptable error range for the particular value.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

All ranges disclosed herein also encompass any and all possible sub-ranges and combinations of sub-ranges thereof. Any listed range can be recognized as sufficiently describing and enabling the same range being broken down into at least equal halves, thirds, quarters, fifths, tenths, and so forth. As a non-limiting example, each range discussed herein can be readily broken down into a lower third, middle third and upper third, and the like. All language such as “up to,” “at least,” “greater than,” “less than,” and the like include the number recited and refer to ranges which can be subsequently broken down into sub-ranges as discussed above. Finally, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Whenever the term “at least,” “greater than,” “greater than or equal to”, or a similar phrase precedes the first numerical value in a series of two or more numerical values, the term “at least,” “greater than,” “greater than or equal to” or similar phrase applies to each of the numerical values in that series of numerical values. For example, “at least 1, 2, or 3” is equivalent to “at least 1, at least 2, and/or at least 3.”

Whenever the term “no more than,” “less than,” “less than or equal to,” “no greater than,” “at most” or a similar phrase, precedes the first numerical value in a series of two or more numerical values, the term “no more than,” “less than,” “less than or equal to,” “no greater than,” “at most,” or similar phrase applies to each of the numerical values in that series of numerical values. For example, “less than 3, 2, or 1” is equivalent to “less than 3, less than 2, and/or less than 1.”

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The phrase “at least one” includes “one”, “one or more”, “one or a plurality” and “a plurality”. The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”. The term “consisting essentially of” refers to the inclusion of recited elements and other elements that do not materially affect the basic and novel characteristics of a claimed combination.

As used herein, a “vector” is a plasmid comprising operably linked polynucleotide sequences that facilitate expression of a coding sequence in a particular host organism (e.g., a bacterial expression vector or a plant expression vector). Polynucleotide sequences that facilitate expression in prokaryotes can include, e.g., a promoter, an enhancer, an operator, and a ribosome binding site, often along with other sequences. Eukaryotic cells can use promoters, enhancers, termination and polyadenylation signals and other sequences that are generally different from those used by prokaryotes.

As used herein, the term “casein micelles” are micelles comprising casein proteins. Examples of casein micelles are described in U.S. patent application Ser. No. 16/741,680 (U.S. Pat. No. 11,326,176), filed on Jan. 13, 2020, titled “Recombinant micelle and method of in vivo assembly,” and in U.S. patent application Ser. No. 17/826,021 filed on May 26, 2022, both incorporated herein by reference in its entirety. Recombinant casein micelles can be made in vivo or in vitro using the methods described therein. U.S. patent application Ser. No. 17/826,021 (United States Patent Application US20220290167A1), titled “Recombinant micelle and method of in vivo assembly” teaches vectors and sequences for making recombinant casein proteins and micelles, which is incorporated herein by reference in its entirety.

As used herein, the term “milk” means a liquid composition that contains soluble casein micelles and where the weight of soluble casein micelles is equal to or greater than 1% of the total protein weight in the composition.

U.S. Pat. No. 11,457,649 describes a substitute dairy food, and U.S. patent application Ser. No. 16/862,011 (Publication No. US20210010017A1) describes food compositions comprising a milk protein, both of which are incorporated herein by reference in their entirety.

As used herein, the term “dairy characteristic” means a characteristic selected from one of the following characteristics of a dairy food: adhesiveness, airiness, appearance, aroma, binding, chewdown, chewiness, coagulation, cohesiveness, compactness, creaminess, crispiness, crumbliness, density, elasticity, emulsification, fattiness, firmness, flavor, foaminess, graininess, greasiness, hardness, handling, juiciness, leavening, mouthcoating, mouthfeel, richness, roughness, slipperiness on tongue, smoothness, springiness, structure, taste, tenderness, texture, thickness, uniformity, and wetness.

In some aspects, the current disclosure provides food products and food product substitutes comprising the nucleic acids disclosed herein. Contemplated food products include dairy products or products that resembles a dairy product (i.e., dairy product substitutes). The term “dairy product” as used herein refers to milk (e.g., whole milk (at least 3.25% milk fat), partly skimmed milk (from 1% to 2% milk fat), skim milk (less than 0.2% milk fat), cooking milk, condensed milk, flavored milk, goat milk, sheep milk, dried milk, evaporated milk, milk foam), and products derived from milk, including but not limited to yogurt (e.g., whole milk yogurt (at least 6 grams of fat per 170 g), low-fat yogurt (between 2 and 5 grams of fat per 170 g), nonfat yogurt (0.5 grams or less of fat per 170 g), greek yogurt (strained yogurt with whey removed), whipped yogurt, goat milk yogurt, Labneh (labne), sheep milk yogurt, yogurt drinks (e.g., whole milk Kefir, low-fat milk Kefir), Lassi), cheese (e.g., whey cheese such as ricotta; pasta filata cheese such as mozzarella; semi-soft cheese such as Havarti and Muenster; medium-hard cheese such as Swiss and Jarlsberg; hard cheese such as Cheddar and Parmesan; washed curd cheese such as Colby and Monterey Jack; soft ripened cheese such as Brie and Camembert; fresh cheese such as cottage cheese, feta cheese, cream cheese, and curd; processed cheese; processed cheese food; processed cheese product; processed cheese spread; enzyme-modulated cheese; cold-pack cheese), dairy-based sauces (e.g., fresh, frozen, refrigerated, or shelf stable), dairy spreads (e.g., low-fat spread, low-fat butter), cream (e.g., dry cream, heavy cream, light cream, whipping cream, half-and-half, coffee whitener, coffee creamer, sour cream, creme fraiche), frozen confections (e.g., ice cream, smoothie, milk shake, frozen yogurt, sundac, gelato, custard), dairy desserts (e.g., fresh, refrigerated, or frozen), butter (e.g., whipped butter, cultured butter), dairy powders (e.g., whole milk powder, skim milk powder, fat-filled milk powder (i.e., milk powder comprising plant fat in place of all or some animal fat), infant formula, milk protein concentrate (i.e., protein content of at least 80% by weight), milk protein isolate (i.e., protein content of at least 90% by weight), whey protein concentrate, whey protein isolate, demineralized whey protein concentrate, demineralized whey protein concentrate, .beta.-lactoglobulin concentrate, .beta.-lactoglobulin isolate, alpha-lactalbumin concentrate, alpha-lactalbumin isolate, glycomacropeptide concentrate, glycomacropeptide isolate, casein concentrate, casein isolate, nutritional supplements, texturizing blends, flavoring blends, coloring blends), ready-to-drink or ready-to-mix products (e.g., fresh, refrigerated, or shelf stable dairy protein beverages, weight loss beverages, nutritional beverages, sports recovery beverages, and energy drinks), puddings, gels, chewables, crisps, and bars. As used herein, the term “food product substitute” (e.g., “dairy product substitute”) refers to a food product that resembles a conventional food product (e.g., can be used in place of the conventional food product). Such resemblance can be due to any physical, chemical, or functional attribute. In some embodiments, the resemblance of the food product provided herein to a conventional food product is due to a physical attribute. Non-limiting examples of physical attributes include color, shape, mechanical characteristics (e.g., hardness, G′ storage modulus value, shape retention, cohesion, texture (i.e., mechanical characteristics that are correlated with sensory perceptions (e.g., mouthfeel, fattiness, creaminess, homogenization, richness, smoothness, thickness), viscosity, and crystallinity. In some embodiments, the resemblance of the food product provided herein and a conventional food product is due to a chemical/biological attribute. Non-limiting examples of chemical attributes include nutrient content (e.g., type and/or amount of amino acids (e.g., PDCAAS score), type and/or amount of lipids, type and/or amount of carbohydrates, type and/or amount of minerals, type and/or amount of vitamins), pH, digestibility, shelf-life, hunger and/or satiety regulation, taste, and aroma. In some embodiments, the resemblance of the food product provided herein to a conventional food product is due to a functional attribute. Non-limiting examples of functional attributes include gelling/agglutination behavior (e.g., gelling capacity (i.e., time required to form a gel (i.e., a protein network with spaces filled with solvent linked by hydrogen bonds to the protein molecules) of maximal strength in response to a physical and/or chemical condition (e.g., agitation, temperature, pH, ionic strength, protein concentration, sugar concentration, ionic strength)), agglutination capacity (i.e., capacity to form a precipitate (i.e., a tight protein network based on strong interactions between protein molecules and exclusion of solvent) in response to a physical and/or chemical condition), gel strength (i.e., strength of gel formed, measured in force/unit area (e.g., pascal (Pa))), water holding capacity upon gelling, syneresis upon gelling (i.e., water weeping over time)), foaming behavior (e.g., foaming capacity (i.e., amount of air held in response to a physical and/or chemical condition), foam stability (i.e., half-life of foam formed in response to a physical and/or chemical condition), foam seep), thickening capacity, use versatility (i.e., ability to use the food product in a variety of manners and/or to derive a diversity of other compositions from the food product; e.g., ability to produce food products that resemble milk derivative products such as yoghurt, cheese, cream, and butter), and ability to form protein dimers.

Where a definition or use of a term in an incorporated reference is inconsistent or contrary to the definition of that term provided herein, the definition of that term provided herein applies and the definition of that term in the reference does not apply.

As used herein, the term “recombinant” refers to nucleic acids or proteins formed by laboratory methods of genetic recombination (e.g., molecular cloning) to bring together genetic material from multiple sources, creating sequences that would otherwise not be found in the genome. Recombinant proteins may be expressed in vivo in various types of host cells, including plant cells, bacterial cells, fungal cells, avian cells, and mammalian cells. Recombinant proteins may also be generated in vitro. As used herein, the term “tagged protein” refers to a recombinant protein that includes additional peptides that are not part of the native protein and that remain after post-translational processing.

As used herein, the term “milk solids” refers to the powder that would be left after milk is dried out and the water is removed.

As used herein, the phrase “essentially free of” is used to indicate the indicated component, if present, is present in an amount that does not contribute, or contributes only in a de minimus fashion, to the properties of the composition. In various embodiments, where a composition is essentially free of a particular component, the component is present in less than a functional amount. In various embodiments, the component may be present in trace amounts. Particular limits will vary depending on the nature of the component, but may be, for example, selected from less than 10% by weight, less than 9% by weight, less than 8% by weight, less than 7% by weight, less than 6% by weight, less than 5% by weight, less than 4% by weight, less than 3% by weight, less than 2% by weight, less than 1% by weight, less than 0.5% by weight, less than 0.1% by weight, or less than 0.05% by weight, or less than 0.01% by weight.

As used herein, the term “stably expressed” refers to expression and accumulation of a protein in a plant cell over time. As an example, a recombinant protein may accumulate because it is not degraded by endogenous plant proteases. As a further example, a recombinant protein is considered to be stably expressed in a plant if it is present in the plant in an amount of 1% or higher per total protein weight of soluble protein extractable from the plant.

As used herein, the term “a detectable amount” refers to an amount of a composition (e.g., a molecule) that can be detected using the most sensitive analytical techniques up to date, including for example, liquid chromatography methods (e.g., reverse phase HPLC, size exclusion, normal phase chromatography), mass spectrometry (e.g., electrospray tandem mass spectrometry, and electrospray FT-ICR mass spectrometry), or a combination of analytical techniques (e.g., liquid chromatography-tandem mass spectrometry (LC-MS/MS)). In some cases, a detectable amount is at a concentration above 10-2 mol/L, 10-3 mol/L, 10-4 mol/L, 10-5 mol/L, 10-6 mol/L, 10-7 mol/L, 10-8 mol/L, 10-9 mol/L, or 10-10 mol/L.

As used herein, the term “naturally occurring” means without genetic modification. For example, a naturally occurring ratio of two plant proteins means a ratio of the two plant proteins found in plant (e.g., plant seed), where the plant is not genetically modified to manipulate the expression levels of the two proteins.

As used herein, the term “recombinant” refers to nucleic acids or proteins formed by laboratory methods of genetic recombination (e.g., molecular cloning) to bring together genetic material from multiple sources, creating sequences that would otherwise not be found in the genome. Recombinant proteins may be expressed in vivo in various types of host cells, including plant cells, bacterial cells, fungal cells, avian cells, and mammalian cells. Recombinant proteins may also be generated in vitro. As used herein, the term “tagged protein” refers to a recombinant protein that includes additional peptides that are not part of the native protein and that remain after post-translational processing.

Definition of standard chemistry terms may be found in reference works, including but not limited to, Carey and Sundberg “Advanced Organic Chemistry 4th Ed.” Vols. A (2000) and B (2001), Plenum Press, New York.

As used herein, the term “homogenous” means of uniform structure or composition throughout, such that individual components (e.g., probiotics, particles) cannot be separately observed with naked eye.

As used herein, the term “plant” includes whole plant, plant organ, plant tissues, and plant cell and progeny of same, but is not limited to angiosperms and gymnosperms such as Arabidopsis, potato, tomato, tobacco, alfalfa, lemice, carrot, strawberry, sugarbeet, cassava, sweet potato, soybean, lima bean, pea, chick pea, maize (com), turf grass, wheat, rice, barley, sorghum, oat, oak, eucalyptus, walnut, palm and duckweed a well as fern and moss. Thus, a plant may be a monocot, a dicot, a vascular plant reproduced from spores such as fern or a nonvascular plant such as moss, liverwort, hornwort and algae. The term “plant,” as used herein, also encompasses plant cells, seeds, plant progeny, propagule whether generated sexually or asexually, and descendants of any of these, such as cuttings or seed. Plant cells include suspension cultures, callus, embryos, meristematic regions, callus tissue, leaves, roots, shoots, gametophytes, sporophytes, pollen, seeds and microspores. Plants may be at various stages of maturity and may be grown in liquid or solid culture, or in soil or suitable media in pots, greenhouses or fields. As used herein, the term “plant protein” refers to a protein that is at least 70% homologous to a protein that naturally occurs in a plant.

As used herein, the term “dicot” refers to a flowering plant whose embryos have two seed leaves or cotyledons. Examples of dicots include Arabidopsis, tobacco, tomato, potato, sweet potato, cassava, alfalfa, lima bean, pea, chick pea, soybean, carrot, strawberry, lettuce, oak, maple, walnut, rose, mint, squash, daisy, quinoa, buckwheat, mung bean, cow pea, lentil, lupin, peanut, fava bean, French beans, mustard, or cactus.

As used herein, the term “monocot” refers to a flowering plant whose embryos have one cotyledon or seed leaf. Examples of monocots include turf grass, maize (corn), rice, oat, wheat, barley, sorghum, orchid, iris, lily, onion, palm, and duckweed.

As used herein, the term “transgenic plant” means a plant that has been transformed with one or more exogenous nucleic acids. “Transformation” refers to a process by which a nucleic acid is stably integrated into the genome of a plant cell. “Stably transformed” refers to the permanent, or non-transient, retention, expression, or a combination thereof of a polynucleotide in and by a cell genome. A stably integrated polynucleotide is one that is a fixture within a transformed cell genome and can be replicated and propagated through successive progeny of the cell or resultant transformed plant. Transformation can occur under natural or artificial conditions using various methods. Transformation can rely on any method for the insertion of nucleic acid sequences into a prokaryotic or eukaryotic host cell, including Agrobacterium-mediated transformation as illustrated in U.S. Pat. Nos. 5,159,135; 5,824,877; 5,591,616 and 6,384,301, all of which are incorporated herein by reference in its entirety. Methods for plant transformation also include microprojectile bombardment as illustrated in U.S. Pat. Nos. 5,015,580; 5,550,318; 5,538,880; 6,153,812; 6,160,208; 6,288,312 and 6,399,861, all of which are incorporated herein by reference in its entirety. Recipient cells for the plant transformation include meristem cells, callus, immature embryos, hypocotyls explants, cotyledon explants, leaf explants, and gametic cells such as microspores, pollen, sperm and egg cells, and any cell from which a fertile plant can be regenerated, as described in U.S. Pat. Nos. 6,194,636; 6,232,526; 6,541,682 and 6,603,061 and U.S. Patent Application publication US 2004/0216189 A1, all of which are incorporated herein by reference in its entirety.

Additional methods and concepts related to codon optimization are described in U.S. Patent Application publication US20200024327A1 (Optimized factor viii gene) to Tan et al and in U.S. Pat. No. 9,427,003 (Synthetic genes) to Larrinua et al, both of which are incorporated herein by reference in its entirety.

As used herein, the term “in-vitro” means outside a living organism.

As used herein, the term “fusion protein” refers to a protein comprising at least two constituent proteins that are encoded by separate genes, and that have been joined so that they are transcribed and translated as a single polypeptide.

Certain aspects of the disclosure have other steps or elements in addition to or in place of those mentioned above. The steps or elements will become apparent to those skilled in the art from a reading of the following detailed description when taken with reference to the accompanying drawings.

While some embodiments of the present invention have been shown and described herein, it will be obvious to those skilled in the art that such embodiments are provided by way of example only. Numerous variations, changes, and substitutions will now occur to those skilled in the art without departing from the invention. Various alternatives to the embodiments of the invention described herein may be employed in practicing the invention. It is intended that the following claims define the scope of the invention and that methods and structures within the scope of these claims and their equivalents be covered thereby.

1-57. (canceled)

58. A nucleic acid molecule comprising a nucleotide sequence selected from SEQ ID NO: 605-SEQ ID NO: 806.

59. The nucleic acid molecule of claim 58, wherein the nucleotide sequence is at least 85%, at least 90%, at least 95%, at least 96%, at least 97%, at least 98%, at least 99%, or 100% identical to any one of SEQ ID NO: 605-SEQ ID NO: 805.

60. The nucleic acid molecule in claim 58, wherein the nucleotide sequence is between 72% and 80% identical to SEQ ID NO: 806.

61. The nucleic acid molecule in claim 58, wherein the nucleotide sequence codes for kappa casein.

62. The nucleic acid molecule of claim 58, wherein the nucleotide sequence is at least 70% but no more than 80% identical to any one of SEQ ID NO: 605-SEQ ID NO: 806.

63. The nucleic acid molecule of claim 58, wherein the nucleotide sequence is at least 72% but no more than 78% identical to any one of SEQ ID NO: 605-SEQ ID NO: 806.

64. The nucleic acid molecule of claim 58, wherein the nucleotide sequence is at least 74% but no more than 76% identical to any one of SEQ ID NO: 605-SEQ ID NO: 806.

65. The nucleic acid molecule in claim 58, wherein the nucleic acid molecule does not comprise at least two, at least three, at least four, at least five, at least six, at least seven, at least eight, at least nine, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, or at least 46 of the sequences selected from the group consisting of GGTACC, ACCGGT, GGGCCC, GTGCAC, GGCGCGCC, GGTACC, CCTAGG, GGATCC, AGATCT, CACGTC, ATCGAT, TTCGAA, ATCGAT, CGGCCG, GAGCTC, GAATTC, GATATC, AAGCTT, GTTAAC, GGTACC, ACGCGT, CCATGG, CATATG, GCTAGC, GCGGCCGC, ATGCAT, TTAATTAA, CTCGAG, GGGCCC, CTGCAG, CGATCG, CAGCTG, GAGCTC, CCGCGG, GTCGAC, CCCGGG, TACGTA, ACTAGT, GCATGC, CTCGAG, CCCGGG, TCTAGA, CTCGAG, CCCGGG, GGTCTC and GAAGAC.

66. The nucleic acid molecule in claim 65, wherein the nucleic acid molecule does not comprise GGTCTC.

67. The nucleic acid molecule in claim 66, wherein the nucleic acid molecule does not comprise GAAGAC.

68. A food composition comprising any one of SEQ ID NO: 605-SEQ ID NO: 806.

69. The food composition of claim 68, wherein any one of SEQ ID NO: 605-SEQ ID NO: 806 codes for kappa casein.

70. A fusion protein, comprising a casein protein and a peptide sequence, wherein the peptide sequence comprises an amino acid sequence that is at least 85%, at least 90%, at least 95%, at least 96%, at least 97%, at least 98%, at least 99%, or 100% identical to SEQ ID NO: 808.

71. The fusion protein in claim 70, wherein the casein protein is alpha S1 casein, alpha S2 casein, beta casein, or kappa casein.

72. The fusion protein of claim 71, wherein kappa casein is encoded by a nucleotide sequence selected from SEQ ID NO: 605-SEQ ID NO: 806.

73. The fusion protein of claim 72, wherein the nucleotide sequence is at least 85%, at least 90%, at least 95%, at least 96%, at least 97%, at least 98%, at least 99%, or 100% identical to any one of SEQ ID NO: 605-SEQ ID NO: 805.

74. The fusion protein of claim 72, wherein the nucleotide sequence is between 72% and 80% identical to SEQ ID NO: 806.

75. The fusion protein of claim 72, the nucleotide sequence is at least 70% but no more than 80% identical to any one of SEQ ID NO: 605-SEQ ID NO: 806.

76. The fusion protein of claim 72, wherein the nucleotide sequence is at least 72% but no more than 78% identical to any one of SEQ ID NO: 605-SEQ ID NO: 806.

77. The fusion protein of claim 72, wherein the nucleotide sequence is at least 74% but no more than 76% identical to any one of SEQ ID NO: 605-SEQ ID NO: 806.
